(19) United States
(12) Patent Application Publication
(10) Pub. No.: US 2011/0152798 A1
(43) Pub. Date: Jun. 23, 2011

(54) Element for facilitating the cutting to size of a dressing for vacuum therapy of a wound

(76) Inventors: Axel Eckstein, Heidenheim (DE); Pierre Croizat, Hebrechtingen (DE); Juergen Hofstetter, Heidenheim (DE)

(21) Appl. No.: 12/968,335

(22) Filed: Dec. 15, 2010

Related U.S. Application Data
(60) Provisional application No. 61/295,188, filed on Jan. 15, 2010.

(30) Foreign Application Priority Data
Dec. 18, 2009 (EP) 09 015 673.8

(51) Int. Cl. A61M 1/00; B32B 38/10

(52) U.S. Cl. 604/319; 156/267

(57) ABSTRACT

A device for use in the vacuum therapy of wounds comprising an air-impermeable cover material for the air-tight closure of the wound and the wound environment, a liquid-permeable wound dressing, a means for functional connection of the wound space to a source of vacuum located outside the cover material, so that a vacuum can be established in the wound space and liquids can be suctioned out of the wound space, and a cutting-to-size element for the liquid-permeable wound dressing.

This application claims Paris convention priority to EP 09 015 673.8 filed on Dec. 18, 2009 as well as benefit to U.S. 61/295,188 filed Jan. 15, 2010, the entire disclosures of which are hereby incorporated by reference.

### BACKGROUND OF THE INVENTION

The invention relates to a device for use in the vacuum therapy of wounds comprising an air-impermeable cover material for the air-tight closure of the wound and the wound environment, and a means for functional connection of the wound space to a source of vacuum outside the cover material so that a vacuum can be established in the wound space and liquids can be suctioned out of the wound space.

A wound is defined as the severing of the connection of tissues of the integumentary system of human beings and animals. It can be associated with a loss of substance.

Devices for the vacuum therapy of wounds are known according to the prior art.

For example, WO1993/009727 describes a device to promote wound healing by the application of a vacuum to the region of the skin containing and surrounding the wound. The device according to WO1993/009727 comprises a vacuum means for producing the vacuum, an air-tight covering of the wound, which is functionally connected to the vacuum means, and a wound dressing for positioning on the wound within the air-tight cover. The dressing is preferably an open-cell polymer foam, for example, polyester foam. The characteristics and size of the dressing can be adapted to the individual wound. As can be seen from the description of WO1993/009727, application of the vacuum therapy can accelerate the healing of different types of wounds, for example, burns, pressure sores, or contused wounds.

During the treatment, vacuum can be continuously maintained until the dressing is changed. Alternatively, the vacuum can be applied during alternating cycles, or cycles with different levels of vacuum can be applied.

Devices for vacuum therapy of wounds are commercially available, for example, the V.A.C.® device from KCI (KCI Medizinprodukte GmbH, Germany). In commercially available devices, a wound dressing is often deployed that contains an open-cell polymer foam, such as polyvinylalcohol (PVA) or polyurethane (PU). In addition to the use of open-cell polymer foam, other materials have been described for the production of wound dressings for the vacuum therapy of wounds. For example, WO2001/89431 describes a collagen matrix as a wound dressing for the vacuum therapy of wounds. GB2415908 describes the use of fibrous material that can also be bioresorbable in wound dressings for the vacuum therapy of wounds.

Wounds can differ in shape and size. From the prior art, it is known that dressings for vacuum therapy are adapted to individual wounds. So, for example, WO1994/20041 states that the dressing for vacuum therapy is cut to a suitable shape and size so that it can be placed within the wound. According to WO1994/20041, the dressing may alternatively be sufficiently large to overlap the surrounding skin. A configuration of the wound dressing that overlaps healthy skin is intended, according to WO1994/20041, to prevent overgrowth of the wound tissue during the therapy. In practice, the wound dressings are frequently inserted in the wound space because, in a configuration that overlaps the wound edge, pressure is applied to the wound edge and to the surrounding undamaged skin. In both of the previous cases, the wound dressing must be adapted to the shape and size of the wound. This is usually done purely by visual judgment.

Means and methods for preparing precisely fitting wound dressings for vacuum therapy are known from prior art.

For instance, for the individual preparation of precisely fitting wound dressings for vacuum therapy, WO2008/57600 suggests dissolving a bioresorbable polymer and a porogenic system in a solvent and, after removal of the solvent, precisely fitting the dressing in the wound space. Using this method, the wound dressing can either be adapted to the size and shape of the wound by manual shaping, or the wound dressing can be formed into the shape of a rope, which is inserted into the wound in a rolled-up form. The porogenic system, which forms pores when it comes into contact with wound exudate, is composed of sodium bicarbonate and citric acid, for example.

WO2005/009488 suggests specially shaped dressings for wounds in exposed positions, for example, on the heel of the foot.

WO2009/097308 describes a cutting device for wound dressings, in particular, for wound dressings for use in vacuum therapy.

Further, wound dressings for vacuum therapy that are intended to facilitate the adaptation to individual wounds are commercially available. So, for example, the V.A.C.® Simplace Dressing Kit™ manufactured by KCI contains the foam dressing GranuFoam™, which can be adapted to a wound size without cutting. The dressing is spiral-shaped and is available in different sizes.

The V.A.C.® GranuFoam™ Standard Dressing Kits from KCI contain foam dressings of different shapes and sizes for different wounds. The dressing V.A.C.® GranuFoam™ Thin Dressing from KCI was developed especially for flat wounds. It has perforations that are intended to facilitate the cutting to size of the dressing.

The object of this invention is to further improve vacuum therapy of wounds and to overcome the disadvantages of the prior art. A further purpose of this invention is to make devices and methods available for the vacuum therapy of wounds with which treatment can be performed as effectively and gently as possible. This object is to be solved in particular by adapting the wound dressing as precisely as possible to the shape of the wound. This is desirable because, if the wound dressing is too small, it may produce unwanted cavities, whereas a dressing that is too large can result in irritation of the wound edges. Individual adaptation of the dressing is to be performed as simply and quickly as possible to shorten the time needed to apply the wound dressing and keep stress to the patient to a minimum. Further, individual adaptation of the dressing is to be performed in as few steps as possible, to ensure the dressing remains sterile. Moreover, for practical purposes, it is desirable if only a minimum number of different wound dressings have to be stocked for application to a large variety of wounds.

### SUMMARY OF THE INVENTION

This object is inventively solved by providing a device for the vacuum therapy of wounds comprising

a) an air-impermeable cover material for the air-tight closure of the wound and the wound environment
b) a means of functionally connecting the wound region to a source of vacuum located outside the cover material, so that a vacuum can be established in the wound space and liquids can be suctioned out of the wound space
c) a liquid-permeable dressing for inclusion in the interstice formed between the wound surface and the cover material, characterized in that the device furthermore comprises
d) a cutting-to-size element for the liquid-permeable dressing, wherein
d1) the cutting-to-size element comprises a transparent film with a first and a second side,
d2) the first side of the transparent film can be written on, so that the outline of the wound can be traced onto the cutting-to-size element,
d3) the transparent film has a self-adhesive coating at least on its second side facing the first side, so that the cutting-to-size element can be adhesively fixed to the side of the wound dressing that faces away from the wound during use.
   as well as by a procedure for the provision of a cut-to-size liquid-permeable wound dressing for the vacuum therapy of wounds, comprising the steps of
   a) providing a cutting-to-size element
   b) providing a liquid-permeable wound dressing
   c) tracing the outline of a wound onto the cutting-to-size element
   d) adhesively fixing of the cutting-to-size element on the side of the liquid-permeable dressing that faces away from the wound during use, so that a flat combination of the wound dressing and cutting-to-size element arises,
   e) cutting to size of the flat combination of the wound dressing and cutting-to-size element, wherein cutting is performed along the outline traced on the cutting-to-size element described in Step c).

In the procedure for providing a liquid-permeable dressing cut to size for the vacuum therapy of wounds, components a) to d) of the aforementioned device for the vacuum therapy of wounds are provided first. Subsequently the wound outline can be traced onto the cutting-to-size element using a suitable pen, for example, a commercially available removable (“non-permanent”) or non-removable (“permanent”) film marker pen. The outline of the wound can be traced onto the cutting-to-size element while the cutting-to-size element is held directly above the wound, for example, at a distance of 2 cm. The wound outline can alternatively be traced while the cutting-to-size element is placed on the wound. Once the wound outline has been traced onto the cutting-to-size element, the cutting-to-size element is adhesively fixed to the side of the liquid-permeable dressing facing away from the wound during use, so that a flat combination comprising the wound dressing and cutting-to-size element arises.

Finally, the combination of the wound dressing and cutting-to-size element can be cut to size along the traced outline of the wound, wherein a sterilized pair of scissors is preferably used for cutting to size.

The liquid-permeable dressing prepared by cutting to size by means of the inventive method has the precise shape of the wound and can be placed on the wound. An inventively prepared dressing cut to size can improve the effectiveness of the vacuum therapy of wounds because neither cavities due to insufficient size of a dressing nor pressure on the wound edges due to an oversized dressing will occur. Furthermore, a precisely fitting dressing cut to size according to the inventive method feels more comfortable for the patient during vacuum therapy because pressure points can be avoided. The inventive method for the preparation of a wound dressing for the vacuum therapy of wounds further enables very quick and simple adaptation of the dressing to the individual shape and size of the wound by the physician or the person performing the treatment. Possibly necessary subsequent adjustment of a dressing already cut to size by visual judgment, as is usual in prior art, is no longer required. This is of benefit to the physician who is often under time pressure when treating wounds. Further, fast medical care of the wound relieves the patient. The inventive method for the fast preparation of a wound dressing for the vacuum therapy of wounds reduces, in a few steps, the risk of contamination of the components of the wound dressing or the open wound.

According to a preferred embodiment, the cutting-to-size element remains on the dressing throughout the vacuum treatment. This has the advantage of enabling wound treatment to be performed quickly in just a few steps. A cutting-to-size element that remains on the wound dressing can also be advantageous to the stabilization of the vacuum dressing, especially in the case of large wounds, for example, wounds that exceed a surface area of 50 cm². A cutting-to-size element that remains on the dressing may also contribute toward a more even distribution of pressure in the wound region. In so doing, a cutting-to-size element that remains on the dressing can also provide protection of the wound from external influences during vacuum therapy.

According to a further preferred embodiment, the cutting-to-size element is removed from the wound dressing before the cut-to-size liquid-permeable wound dressing is applied to the wound. Removal of the cutting-to-size element from the cut-to-size dressing after it has been individually adapted can be advantageous if additional layers are not desired in the wound space. The removal of the cutting-to-size element from the cut-to-size dressing may be particularly desirable if a particularly soft wound cover is required. This may be the case with smaller wounds, for example, wounds with a surface area of less than 50 cm².

The inventive device comprises a cover material for the air-tight closure of the wound. The term “air-tight closure” is not intended to imply that no exchange of gases between the wound space and the wound environment takes place. In fact, “air-tight closure” in this context means that the vacuum necessary to perform the vacuum therapy of wounds can be maintained with the vacuum pump intended for this task. Cover material that exhibits slight gas permeability can therefore also be used provided the vacuum required for the vacuum therapy can be maintained.

The air-impermeable cover material can, for example, have the form of a shell made of solid material or the form of a flexible film. Combinations of fixed frames or contact plates with flexible films are also conceivable. In a preferred embodiment of the invention, the cover material for the air-tight closure of the wound comprises a water-insoluble polymer, or metal foil.

In an especially preferred embodiment of the invention, the water-insoluble polymer may be polyurethane, polyester, polypropylene, polyethylene, polyamide, polyvinyl chloride, polyorganosiloxane (silicone) or a mixture of these.

Further suitable polymer film materials are known to persons skilled in the art.

Finished products that exhibit the above-mentioned characteristics can also be used as cover material.

A particularly suitable cover material for the inventive device has proven to be the polyurethane film of the brand Hydrofilm® (Paul Hartmann AG, Germany) or Visulin® (Paul Hartmann AG, Germany).

The cover material is attached in the wound environment or at the wound edge in such a way as to ensure air-tight closure of the wound. In this case, it can be advantageous if the cover material is provided with self-adhesive properties across its entire surface or has a self-adhesive edge. Alternately, adhesion and sealing can be provided by an adhesive film, by a liquid adhesive, or by a sealing compound.

In a preferred embodiment of the invention, the cover material comprises a film made of one or more water-insoluble polymers, wherein the film is provided with self-adhesive properties over its entire area or has a self-adhesive edge.

However, it is also possible for the cover material to adhere to the wound merely by the vacuum created during the vacuum therapy.

In a preferred embodiment, the cover material and the means for functional connection of the wound space to a source of vacuum located outside the cover material are provided ready for use and already connected. It is highly preferred if this embodiment contains a film comprising one or more water-insoluble polymers with a self-adhesive edge, as this configuration makes it considerably easier to apply the dressing.

The inventive device for vacuum therapy comprises a means for functional connection of the wound space to a source of vacuum located outside the cover material, so that a vacuum can be established in the wound space and liquids can be suctioned out of the wound space.

In connection with the invention, the expression “vacuum in the wound space” denotes an air pressure that is rendered lower within the wound dressing than the ambient air pressure (atmospheric air pressure).

“Within the wound dressing” refers to the interstice formed between the cover material and the wound. “Vacuum” is frequently also referred to as “negative pressure.”

The pressure difference between the air pressure within the wound dressing and the ambient air pressure is expressed in mm Hg (millimeters of mercury) in connection with the invention because this is customary in the field of vacuum therapy. 1 mm Hg corresponds to one Torr or 133.322 Pa (Pascal). In connection with the invention, the vacuum, that is, the pressure difference between the air pressure inside the wound dressing and the ambient air pressure is expressed as a positive numerical value in mm Hg.

In one embodiment of the invention, vacuum refers to a vacuum of no more than 500 mm Hg. This vacuum range of no more than 500 mmHg has proved to be conducive to wound healing. In a preferred embodiment of the invention, the vacuum refers to a vacuum of at least 10 mm Hg and no more than 150 mm Hg.

In two further alternative embodiments, each of them preferred, the vacuum a) is a constant vacuum or b) a time-variable vacuum.

“Constant vacuum” (a) in this case refers to the fact that the vacuum is kept essentially constant during the treatment. “Essentially constant” means that, during the treatment, slight upward or downward changes in the vacuum, for example, by 15%, may occur.

A preferred constant vacuum is the range from at least 80 mm Hg to no more than 150 mm Hg.

“Time-variable vacuum” (b) is, in this case, understood to mean that the vacuum undergoes specific variations during the treatment. Specific variations of the air pressure mean those variations of the air pressure that occur when a first target value for the vacuum has been reached after the vacuum dressing has been applied. By contrast, the initial rise phase of the vacuum that occurs after application of the dressing until the first target value has been reached is not included in the term “time-variable vacuum.” This applies analogously to the increase in air pressure to ambient air pressure that is required at the end of the treatment, which is also not covered by the term “time-variable vacuum”.

The lower limit for the “time-variable vacuum” is the ambient air pressure and the upper limit is a maximum vacuum of 500 mm Hg, preferably 150 mm Hg, in particular, 125 mm Hg. The actual vacuum applied during the treatment varies within this range defined by its limit values. The “time-variable vacuum” therefore includes, for example, a single change or multiple changes between one or more higher vacuum values and one or more lower vacuum values. Similarly, “time-variable vacuum” includes a specific single change or multiple changes occurring during the treatment between ambient pressure and one or more higher vacuum values.

In a preferred embodiment, the maximum vacuum for a time-variable vacuum is 125 mm Hg. In this embodiment, the lower limit for the variation of the vacuum is the ambient air pressure. During therapy, the vacuum varies between or within these limit values.

In a further preferred embodiment, the maximum vacuum for a time-variable vacuum is 125 mm Hg. The lower limit for the variation of the vacuum in this embodiment is 20 mm Hg. During the therapy, the vacuum varies between or within these limit values.

In the two embodiments described above, the change between the upper and the lower pressure value can be periodic or non-periodic. A periodic change is preferred. The time intervals in which the higher vacuum and in which the lower vacuum or ambient pressure is maintained can be of different durations. A lower vacuum is preferably maintained for longer than a higher vacuum. Suitable time intervals in which a certain vacuum setting or the ambient pressure is maintained are, for example, 1 min, 2 min, 5 min, 10 min, 30 min, 1 h, 12 h, or 24 h.

A varying vacuum with the parameters stated below is especially preferred, wherein alternation between the two vacuum values is performed continually at the specified time intervals during the therapy: A vacuum of 125 mm Hg for 2 min,

thereafter
a vacuum of 20 mm Hg for 5 min.

The inventive device for vacuum therapy of wounds further comprises a means for the functional connection of the wound space to a source of vacuum located outside the cover material.

The functional connection can be established, for example with a connection tube or with a vacuum connection element. Vacuum connection elements are also known to persons skilled in the art by the term “port.”

In one embodiment, the means for functional connection of the wound space with a vacuum source located outside the cover material is at least one connecting line. The at least one connecting line can be brought through the cover material.

Alternatively, the at least one connecting line can be passed under the edge of the cover material.

In both cases, the penetration point must be sealed air-tight so that the required vacuum can be maintained in the dressing. Suitable sealing means include an adhesive film, adhesive compound, or adhesive strip. The connecting line can, for example, be a tube, a pipe, or another body with a hollow interior. One suitable tube is, for example, a silicone drainage tube.

The connecting line advantageously has a vacuum adapter at the end that is located outside the wound dressing to be connectable with the further components of the vacuum system.

The connecting line has an opening at the end that is located inside the wound dressing.

In a further embodiment, the means for functional connection of the wound space with a vacuum source outside the cover material is a vacuum connection element (port) that can be attached to the inside or outside of the cover material, wherein the cover material has suitable openings. In either case, air-tight sealing either of the penetration opening (port inside) or the contact surface (port outside) must be ensured. Sealing can be achieved, for example, with an adhesive film, with an adhesive compound, or with an adhesive strip. It is also conceivable that the port itself has appropriate fastening means such as adhesive surfaces.

Suitable vacuum connection elements are available commercially. These are typically vacuum connection elements that are adhesively fixed to the outside of the cover material.

The vacuum connection element also advantageously has a vacuum adapter to enable connection to the further components of the vacuum system.

The inventive device for the vacuum therapy of wounds also has a liquid-permeable wound dressing. Any wound dressing according to the prior art can be used as the wound dressing, provided that wound exudate can pass through it and the material exhibits no tendency to grow together with or adhere to the wound tissue. The passage of wound exudate can be ensured by the wound dressing being made of a material that is permeable to liquids. This can, for example, be the case for wound dressings made of foam material. The passage of wound exudate can also be ensured if the wound dressing has suitable openings or channels. Furthermore, it should be possible to cut the dressing to size with a pair of scissors, preferably, with a pair of sterilized scissors.

An especially suitable wound dressing made of a structured gel is disclosed in the German patent application number 102008062472.1. There, a wound dressing for vacuum therapy is described comprising a substrate layer and a wound dressing applied to the side of the substrate layer facing the wound, wherein the wound dressing has openings passing through the wound dressing that are transverse with respect to its surface extent, characterized in that the side of the wound dressing facing the wound has protuberances and recesses and the wound dressing forms a contact surface with a skin surface only in the region of the protuberances.

The wound dressings described in the German patent application numbers DE102008031183.9 and DE102008031182.0 can also be used as a liquid-permeable wound dressing for the inventive device. The wound dressing layers described in DE102008031183.9 and DE102008031182.0 are especially suitable for use in the granulation phase and in the epithelization phase. They comprise a hydrophilic polyurethane foam with a water component of at least 10 percent by weight.

Similarly, the wound dressing can comprise a permeable nonwoven layer or lattice tulle. The permeable nonwoven layer or the tulle preferably consists of a hydrophobic material, for example, polyester. The tulle can further be provided with an ointment.

The ointment compresses Hydrotüll® and Atrauman® (Paul Hartmann AG, Germany) are especially suitable wound dressings.

The problem of possible growing together or adhesion of the wound dressing to the wound tissue is solved in an alternative embodiment of the invention by having the wound dressing either completely consist of a bioresorbable material or providing it with bioresorbable material on the wound side. Suitable bioresorbable materials are known, for example, from DE19609551 or WO02/072163.

Preferably, in particular for the use of the inventive device in the treatment of infected wounds, the wound dressing has an anti-microbial coating. The anti-microbial coating is preferably a silver coating.

An ointment compress with a silver coating that is suitable as a wound dressing layer is the commercial product Atrauman Ag® (Paul Hartmann AG, Germany).

The wound dressing layer can contain an antibiotically active pharmaceutical substance.

The wound dressing can also contain substances that promote wound healing and that are released to the surface of the wound during treatment. One example of such substances are growth factors.

In a further embodiment of the invention, the device for vacuum therapy of wounds comprises at least one additional wound dressing layer, which is applied to the side of the first liquid-permeable dressing facing the wound. This also comprises layers of different materials. The additional wound dressing layer or layers should additionally be made to adhere to the first wound dressing so that they can simultaneously be cut to size. The adhesive connection is intended to prevent the first wound dressing and the additional wound dressing layer from slipping relative to each other. Additionally, if use of more than one additional wound dressing layer is intended, the additional wound dressing layers should also be made to adhere to each other. A combination of multiple layers permits optimum adaptation to the particular wound situation. For example, it can be advantageous to use a combination of a silver-coated ointment compress with a foam dressing as the wound dressing in an infected wound.

The inventive device for the vacuum therapy of wounds also comprises a cutting-to-size element for the liquid-permeable wound dressing. The cutting-to-size element comprises a transparent film with a first and a second side. The first side of the transparent film can be written on, for example, with a commercially available film marker pen. As it is possible to write on the film, the precise outline of the wound to be treated can be traced onto the cutting-to-size element. The transparent film must be sufficiently transparent to allow the outline of the wound to be seen when the film is held against the wound. In particular, it should be possible to see the outline when the film is held closely, for example, at a distance of approx. 2 cm, above the wound. Preferably, the transparent film is also sufficiently transparent to see the outline of the wound when the film is placed on the film. A person skilled in the art can define the transparency by selecting an adequate material and thickness of the film. A thickness of the transparent film of 20 μm to 500 μm, in particular, 25 μm to 100 μm, has proven particularly well suited. The transparent film is preferably a polymer film, in particular, a film made of polyurethane, polyester, polypropylene, polyethylene, polyamide, polyvinyl chloride, polyorganosiloxane (silicone) or a mixture of these. According to an especially preferred embodiment, the transparent film is made of polyurethane film. On the second side opposite the first side, the cutting-to-size element comprises a self-adhesive coating. The self-adhesive coating allows the cutting-to-size element to be adhesively fixed to the side of the wound dressing facing away from the wound during use. The self-adhesive coating preferably comprises a polyacrylate adhesive.

According to a preferred embodiment, the cutting-to-size element further comprises at least one transparent cover film on the side facing the wound during use. The cover film thereby protects the self-adhesive coating of the transparent film. In this embodiment, it is also essential for the combination of transparent film and transparent cover film to exhibit sufficient total transparency to make it possible to see the outline of the wound through the combination when the combination is held up to the wound and/or is placed on the wound. Suitable materials for the transparent cover film include polyethylene, polyester, polypropylene, polyethylene, polyamide, polyvinyl chloride, polyorgnosiloxane, or a combination of these. It has been shown that a siliconized polyester film is particularly suitable as a transparent cover film.

The cutting-to-size element preferably comprises two separate transparent cover film components that are disposed in such a way that they can be pealed away from the transparent film in a simple manner, for example, by the formation of at least one gripping strip. A gripping strip can be formed, for example, by folding back one component of the cover film. The other cover film component can also comprise a gripping strip formed by folding back. Alternatively, the other cover film component can at least partially overlap the gripping strip of the first cover film component.

According to a further advantageous embodiment, the cutting-to-size element comprises at least one additional film on the side of facing away from the wound during use, which preferably comprises a transparent polyester film.

The at least one additional film can be constituted as a protective film. The protective film can be removed after the cutting-to-size element has been taken out of the preferably sterile packaging.

The at least one additional film can, in particular, comprise a transparent polyester film with a first and a second side, wherein the second side of the transparent polyester film is applied to the first side of the transparent film. In this case, the additional film can be left in place while the outline of a wound is traced onto the transparent film. In this embodiment, the outline of the wound is traced on the first side of the transparent polyester film, while the first side of the transparent film is not written on. A polyester film whose second side is placed on the first side of the transparent film can contribute toward stabilizing and protecting the cutting-to-size element and facilitate handling the cutting-to-size element.

In wound therapy, it can be desirable to document the progress of wound healing. As part of wound documentation, it is preferable to record the size and outline of the wound at the beginning of the vacuum therapy. The inventors of this invention have ascertained that the cutting-to-size element according to a further embodiment not only permits the preparation of individually cut to size wound dressings, but can also be used for simple and fast documentation of the size and outline. According to this advantageous embodiment, the cutting-to-size element comprises a further transparent film with a first and a second side, wherein a transparent polyester film is particularly suitable. This further transparent film can be written on its first side, so that the outline of the wound can be traced onto the further transparent film. Furthermore, the further transparent film has a self-adhesive coating on the second side facing the first side, so that the second side of the further transparent film can be adhesively fixed to the first side of the transparent film. According to this embodiment, the outline of the wound is traced onto the first side of the further transparent film. The combination of the first and further transparent film is then adhesively fixed to the liquid-permeable wound dressing. After the combination of wound dressing, transparent film and further transparent film has been cut to size, the further transparent film is pealed away from the transparent film. The further transparent film replicates the shape of the wound outline and can be adhesively fixed, for example, in a patient file.

Preferably, the at least one further film is constituted in two parts, wherein the two parts are disposed in such a way that they can be pealed away from the transparent film in a simple manner, for example, by forming at least one gripping strip. This can be achieved, for example, by a first part of the further film overlapping the second part of the further film.

It has proven particularly advantageous for the cutting-to-size element on the first side to comprise at least one further film and on its second side to comprise at least one transparent cover film. It is also possible for the cutting-to-size element to comprise at least one further film on the side facing away from the wound during use, and to comprise no further layer apart from the self-adhesive coating on its side facing the wound during use.

Further, the invention provides a ready-to-use set for vacuum wound therapy comprising an air-impermeable cover material for air-tight closure of the wound and the wound environment, a means for functional connection of the wound space to a source of vacuum located outside the cover material so that a vacuum can be established in the wound space and liquids can be suctioned out of the wound space, a wound cover made of a liquid-permeable material for inclusion in the interstice formed between the wound surface and the cover material, and a cutting-to-size element for a liquid-permeable wound dressing for the vacuum therapy. The components contained in the set should be packaged in a sterile condition. In particular, the cutting-to-size element, the dressing, the cover material, and the means for functional connection of the wound space to a source of vacuum located outside the cover material should be packaged in a sterile condition. Preferably, all components for which it is medically necessary are packaged in a sterile manner.

The set can contain further optional components, such as, for example, one or more wound dressing layers, adhesion means for fixing the dressing, sealing means for achieving air-impermeable sealing of the dressing, pressure sensors, connecting elements for pressure sensors, additional tubes, connection elements for tubes, disinfection means, skin care means, pharmaceutical preparations, or instructions for use. Especially advantageous components of the set are further a pair of sterilized scissors and a sterilized marker pen. The marker pen that can be optionally included in the set is, in particular, a film marker pen that is suitable for tracing the outline of the wound on the cutting-to-size element.

This set preferably further comprises a ready-to-use vacuum unit. The vacuum unit can contain components such as a pump, one or more liquid vessels, a control unit, a power supply unit, electrical connection means, and tubes. The vacuum unit can also contain a device for the functional connection of the vacuum dressing to an existing stationary source of vacuum.

The advantage of ready-to-use sets is that the vacuum dressing can be applied in a standardized and uncomplicated manner. A further advantage is that all components of the set used in the region of the wound can be provided already sterilized.

In a further preferred embodiment, a cutting-to-size element is provided for use in vacuum therapy in the treatment of a wound produced by a skin grafting. The application comprises the treatment of wounds produced by split-thickness grafts and by full-thickness grafts by means of vacuum therapy. Advantageous effects result from providing a wound dressing whose outline corresponds exactly to the outline of the graft. This makes very even distribution of pressure possible. By using the cutting-to-size element in the treatment of a wound produced by skin grafting, the skin graft can be sufficiently fixed while avoiding harmful shearing forces.

A further object of the invention is a method for vacuum therapy of wounds comprising the steps

a) provision of a device comprising an air-impermeable cover material for the air-tight closure of the wound and the wound environment, a means for functional connection of the wound space to a vacuum source located outside the cover material so that a vacuum can be established in the wound space and liquids can be suctioned out of the wound space, a liquid-permeable dressing for inclusion in the interstice formed between the wound surface and the cover material, characterized in that the device further comprises a cutting-to-size element for the liquid-permeable wound dressing. The cutting-to-size element comprises a transparent film with a first and a second side. The first side of the transparent film can be written on, so that the outline of the wound can be traced onto the cutting-to-size element. Furthermore, at least on its second side facing the first side, the transparent film has a self-adhesive coating so that the cutting-to-size element can be adhesively fixed to the side of the wound dressing that faces away from the wound during use.
b) placing the cutting-to-size element on the wound or alternatively holding the cutting-to-size element at a close distance, for example, at a distance of 2 cm, above the wound.
c) tracing a wound outline
d) adhesively fixing the cutting-to-size element to the side of a liquid-permeable dressing facing away from the wound,
e) cutting the liquid-permeable wound dressing to size with the cutting-to-size element, wherein the outline traced in Step c) is followed when cutting to size
f) placing the cut-to-size liquid-permeable wound dressing onto the wound
g) applying the remaining components of the vacuum dressing to the wound
h) producing of a vacuum of no more than 500 Hg in the wound space for at least 30 minutes and no longer than 7 days.

The cutting-to-size element in Step f) preferably remains on the side of a cut-to-size liquid-permeable wound dressing facing away from the wound during use. However, according to an alternative embodiment, it can be pealed away from the cut-to-size wound dressing before the latter is placed on the wound.

Below the inventive device for the vacuum therapy of wounds is explained in more detail using drawings. However, the invention is not to be understood as limited to the embodiments illustrated in the drawings or in the description of the drawings. Rather the inventive device also comprises combinations of the individual characteristics of the various forms.

### BRIEF DESCRIPTION OF THE DRAWING

FIG. 1 schematic view of one of the vacuum dressings placed on the wound (side view);

FIG. 2 schematic view of an embodiment of the cutting-to-size element (side view);

FIG. 3 schematic view of a further embodiment of the cutting-to-size element (side view);

FIG. 4 schematic view of a further embodiment of the cutting-to-size element (side view); and

FIG. 5 schematic view of a preferred embodiment of a device for the vacuum therapy of wounds applied to a wound (side view)

### DESCRIPTION OF THE PREFERRED EMBODIMENT

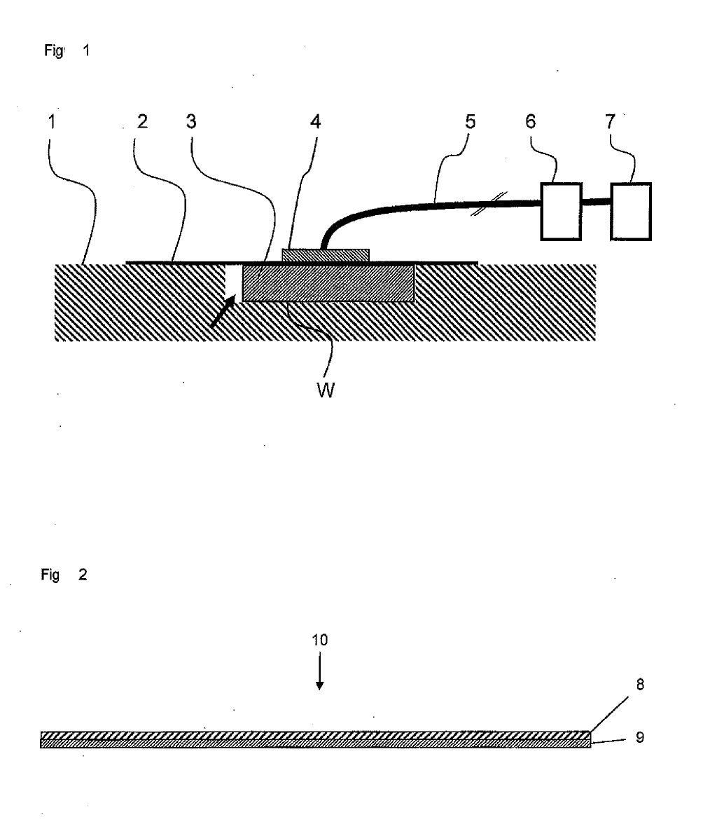

FIG. 1 shows a typical structure of a vacuum dressing applied to the wound. Devices of this type are known in many variations from prior art. The device comprises an air-impermeable cover material 2, a means for functional connection of the wound space to a source of vacuum located outside the cover material 7, a collection vessel for the wound exudate 6, and a liquid-permeable dressing 3, which is located in the wound space. Usually, the liquid-permeable dressing is cut to size by visual judgment. If a wound dressing that is insufficiently adapted to the size and shape of the wound does not fully fill the wound space, cavities can occur between the base of the wound W and the cover material 2 (indicated by an arrow in FIG. 1). Furthermore, a wound dressing which is too large may overlap the wound edge (not depicted in FIG. 1). The means for functional connection of the wound space to a source of vacuum located outside the cover material 7 comprises, in the example depicted here, a vacuum connection element 4, which is termed “port,” and a vacuum connection tube 5. In the example shown here, the port 4 is located on the outside of the air-impermeable cover material 2 facing away from the wound. For functional connection of the wound space with a vacuum unit 7 located outside the cover material, one or more openings (not shown) through the cover material 2 must be provided in this configuration in the region of the vacuum connection element 4. Furthermore, air-tight sealing must be ensured. Such a sealing can be created, for example, by applying a film (not shown in FIG. 1) to the upper side of the port facing away from the wound, which is adhesively fixed to the cover material 2. Application of the dressing can be facilitated if a port is used that already includes a fixing and a sealing means for fixing the port to the cover material. A collection vessel 6 is located between the vacuum connection element 4 and the vacuum unit 7. The cover material 2 is fixed in the wound environment 1, where, as a rule, the skin is not damaged. The cover material should be dimensioned such that the cover material can be fixed outside the area of the wound in the wound environment 1. The cover material 2 can have different dimensions and shapes, for example, circular, oval, or rectangular. It can also be provided in an irregular shape, fitted to the wound. The cover material 2 can be an opaque material, a partially transparent material, or a completely transparent material. Use of transparent material can be advantageous for inspecting the progress of wound healing. Use of only partially transparent or opaque material can be advantageous in that it spares the patient the sight of the wound. Alternatively, the cover material 2 can also be a rigid material that is applied over the wound region in the form of a shell that is open toward the wound and is attached in the region surrounding the wound 1. The cover material 2 must be attached in the region surrounding the wound 1. This can be achieved by providing the cover material 2 with an adhesive edge. The adhesive edge should be protected by protective strips right up until the dressing is applied. Alternatively, an adhesive substance can be applied either to the edge of the cover material 2 and/or to the undamaged skin in the region surrounding the wound. This has the advantage that adaptation of the cover material to the shape and size of the wound is easier. Attachment and air-tight sealing of the device can also be achieved by the use of adhesive strips or an adhesive compound. Applying the dressing can be made easier by using a port in which a suitable attachment and sealing means is provided to attach the port on the cover material. This is, for example, the case for the commercially available PPM-Drainageport from Phametra-Pharma and Medica-Trading GmbH (Germany).

FIG. 2 shows a side view of an embodiment of the cutting-to-size element 10. The cutting-to-size element 10 comprises a transparent film 8, whose first side can be written on, so that the outline of a wound can be traced onto it. Once the outline of a wound has been traced onto the first side of the transparent film 8, the cutting-to-size element 10 is adhesively fixed to the liquid-permeable wound dressing. Towards this end, the transparent film 8 has a self-adhesive coating 9 on its second side. The self-adhesive coating 9 provides an easy means of adhesive fixing the cutting-to-size element 10 to the wound dressing 3. Subsequently, the liquid-permeable wound dressing 3 can be cut to size together with the cutting-to-size element 10, wherein the cutting action follows the outline traced onto the first side of the transparent film 8.

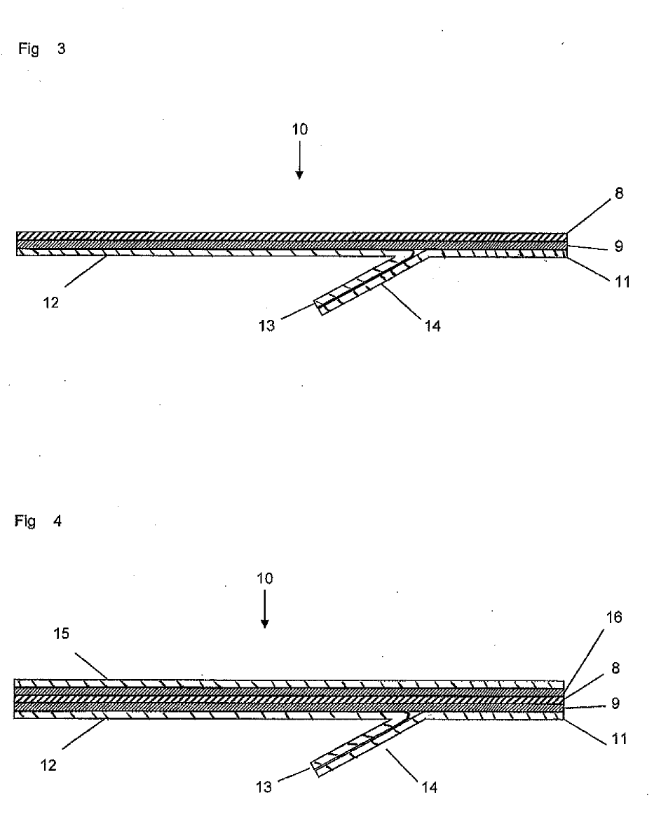

FIG. 3 shows the structure of a further preferred embodiment of the cutting-to-size element 10. The device differs from the device depicted in FIG. 2 in that the self-adhesive coating 9, which is located on the second side of the transparent film 8, is covered by the transparent cover films 11 and 12. Alternatively, the cutting-to-size element 10 can merely comprise a transparent cover film, or even more than two transparent cover films (not depicted). The transparent cover films 11 and 12 depicted in FIG. 3 comprise the gripping strips 13 and 14 that are formed by folding back (gripping strip 13) and/or overlapping the other transparent film (gripping strip 14). The gripping strips 13 and 14 facilitate removal of the transparent cover films 11 and 12. Other methods with which removal of at least one transparent cover film can be facilitated are known to persons skilled in the art.

A cutting-to-size element that comprises at least one transparent cover material is especially suitable for use in the inventive procedures. On the one hand, the at least one transparent cover film facilitates handling of the cutting-to-size element 10 because the adhesive surface 9 is covered. Furthermore, the at least one transparent cover film stabilizes the cutting-to-size element 10 so that it does not break off so easily. Moreover, the at least one transparent cover film protects the adhesive surface 9 of the transparent film 8 from wound exudate. Contact with wound exudate can accidentally occur, for example, when the cutting-to-size element is held close to the wound. Contact with wound exudate occurs necessarily when the cutting-to-size element 10 is placed on the wound to trace the wound outline. It is therefore advantageous to detach the at least one transparent cover film from the cutting-to-size element only once the cutting-to-size element has been adhesively fixed to the wound dressing 3.

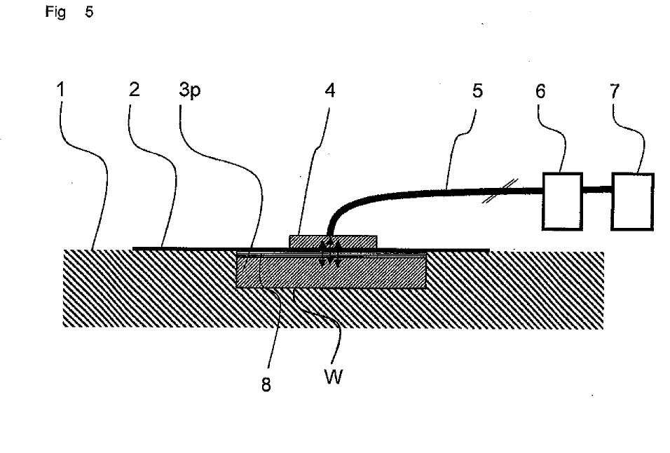

FIG. 4 shows an especially advantageous embodiment of the cutting-to-size element 10. In this embodiment, the transparent film 8 further comprises a transparent polyester film 15 on its first side, which is provided with a self-adhesive coating 16. The transparent polyester film 15 can also be constituted as multiple parts (not depicted in FIG. 4). Further, one or more gripping strips can be attached to the transparent polyester film 15 to facilitate the pealing away of the polyester film 15 from the transparent film 8 (not represented in FIG. 4). The removal of the polyester film 15 could additionally be facilitated by folding back (not depicted in FIG. 4). Other methods with which removal of at least one of the transparent polyester films can be facilitated are known to persons skilled in the art.

One advantage of the embodiment shown in FIG. 4 is that the transparent polyester film can initially be left on the transparent film 8 during application. This allows the wound outline to be traced directly onto the transparent polyester film 15. The transparent polyester film 15 can be pealed away from the transparent film 8 after the wound dressing has been cut to size and archived as a documentation aid for documenting the wound size.

FIG. 5 shows a preferred embodiment of a device applied to a wound for the vacuum therapy of wounds where the liquid-permeable wound dressing 3p has already been cut to size. The device comprises an air-impermeable cover material 2, the means 4 and 5 for functional connection of the wound space to a source of vacuum 7 located outside the cover material, a collection vessel for the wound exudate 6, and a cut-to-size liquid-permeable wound dressing 3p that is located in the wound space at the base of the wound W. According to the embodiment depicted in FIG. 5, after the combination of transparent film 8 and wound dressing 3 has been cut to size, the transparent film 8 remains on the cut-to-size wound dressing 3p. The combination of transparent film 8 and wound dressing 3p that has been precisely cut to the size and outline of the individual wound is inserted in the wound space. The wound space is closed with an air-impermeable cover material 2. For functional connection of the wound space to a vacuum unit 7 located outside the cover material, there must be one or more openings (indicated by the double-headed arrow in FIG. 5) through the cover material 2 and the transparent film 8 in this configuration in the region of the vacuum connection element 4. Usually, these openings are made after applying the air-impermeable cover material 2. For this, at least one opening can be cut with a sharp implement, for example, a sterilized scalpel, through the cover material 2 and the transparent film 8 within the area covered by the vacuum connection element 4. The at least one opening can, for example, be applied in the form of one or more cross-shaped slits. However, it is especially advantageous if the openings are cut in the shape of one or a few (for example, 2 to 5) circular openings, each of diameter 3 to 8 mm, through the cover material 2 and the transparent film 8. The cut-out film material should, as far as possible, be removed from the dressing as it might otherwise block the vacuum connection element during vacuum therapy.

### LEGEND FOR THE FIGURES

W Base of wound
1 Wound environment (i.e. usually undamaged skin)
2 Air-impermeable cover material
3 Liquid-permeable dressing placed in a wound
3p Cut-to-size liquid-permeable wound dressing
4 Vacuum connection element (port)
5 Vacuum connection tube
6 Collection vessel
7 Vacuum unit
8 Transparent film
9 Self-adhesive coating on transparent film 8
10 Cutting-to-size element
11, 12 Transparent cover films
13, 14 Gripping strips on transparent cover film, where the gripping strip 13 is formed by folding back the cover film 12, and the gripping strip 14 is constituted by the component of the cover film 11 overlapping the gripping strip 13
15 Application film
16 Self-adhesive coating on application film 15

We claim:

1. A device for the vacuum therapy of a wound, the device comprising:
an air-impermeable cover material for air-tight closure of the wound and of a wound environment;
an element for functional connection of a wound space to a source of vacuum located outside said cover material, so that a vacuum can be established in the wound space and such that liquids can be suctioned out of the wound space;
a liquid-permeable wound dressing for inclusion in an interstice formed between a wound surface and said cover material; and
a cutting-to-size element for said liquid-permeable wound dressing, said cutting-to-size element having a transparent film with a first and a second side, said first side of said transparent film being structured to permit writing thereon, so that an outline of the wound can be traced onto said transparent film, wherein said transparent film has, at least on a second side thereof facing said first side, a self-adhesive coating, so that said cutting-to-size element can be adhesively fixed to a side of said wound dressing facing away from the wound during use.

2. The device for the vacuum therapy of wounds of claim 1, wherein said transparent film is coated on said second side with a polyacrylate adhesive.

3. The device for the vacuum therapy of wounds of claim 1, wherein said transparent film is a polymer film.

4. The device for the vacuum therapy of wounds of claim 3, wherein a polymer of said polymer film comprises polyurethane, polyester, polypropylene, polyethylene, polyamide, polyvinyl chloride, polyorgnosiloxane (silicone) or a mixture of these.

5. The device for the vacuum therapy of wounds of claim 1, wherein said cutting-to-size element further comprises at least one transparent cover film on a side facing the wound during use.

6. The device for the vacuum therapy of wounds of claim 5, wherein said at least one cover film comprises a siliconized polyester film.

7. The device for the vacuum therapy of wounds of claim 1, wherein said cutting-to-size element comprises at least one further film on a side facing away from the wound during use.

8. The device for the vacuum therapy of wounds of claim 7, wherein said at least one further film comprises a transparent polyester film having a first and a second side, wherein said first side of said transparent polyester film is structured to permit writing thereon so that the outline of the wound can be traced onto said transparent polyester film, said transparent polyester film also having a self-adhesive coating on said second side facing said first side, wherein said transparent polyester film can thereby be adhesively fixed to a first side of the transparent film and, after cutting to size of the wound dressing, said transparent polyester film can furthermore be pulled off said transparent film and archived as a documentation aid for documenting a wound size.

9. A method for provision of a cut-to-size liquid-permeable wound dressing for vacuum therapy of wounds using the cutting-to-size element of claim 1, the method comprising the steps of:
a) providing a liquid-permeable wound dressing;
b) tracing an outline of the wound on the cutting-to-size element;
c) adhesively fixing the second side of the cutting-to-size element onto a side of the liquid-permeable wound dressing that faces away from the wound during use, so that a flat combination of the wound dressing and the cutting-to-size element arises; and
d) cutting to size the flat combination of the wound dressing and cutting-to-size element produced in step c), wherein a cutting action is performed along the outline traced onto the cutting-to-size element in step b).

10. The method for provision of a cut-to-size liquid-permeable wound dressing for vacuum therapy of wounds of claim 9, further comprising the step of removing the cutting-to-size element from the cut-to-size wound dressing.

11. The method for provision of a cut-to-size liquid-permeable wound dressing for vacuum therapy of wounds of claim 9, wherein the wound outline is traced onto the cutting-to-size element while the cutting-to-size element is held close to or placed onto the wound.

12. Use of the device of claim 1 for vacuum for therapy of wounds.

13. Use of the method of claim 9 for vacuum therapy of wounds.

14. A method for vacuum therapy of a wound, the method comprising the steps of:
a) providing an air-impermeable cover material for air-tight closure of the wound and of a wound environment;
b) providing an element for functional connection of a wound space to a source of vacuum located outside the cover material, so that a vacuum can be established in the wound space and such that liquids can be suctioned out of the wound space;
c) providing a cutting-to-size element for the liquid-permeable wound dressing, wherein the cutting-to-size element has a transparent film with a first and a second side, the first side of the transparent film structured to permit writing thereon such that an outline of the wound can be traced onto the transparent film, the transparent film also having, at least on a second side thereof facing the first side, a self-adhesive coating, wherein the cutting-to-size element can be adhesively fixed to a side of a wound dressing facing away from the wound during use;
d) providing a liquid-permeable wound dressing;
e) tracing a wound outline on the cutting-to-size element;
f) adhesively fixing the cutting-to-size element onto a side of the liquid-permeable wound dressing facing away from the wound during use, so that a flat combination of the wound dressing and cutting-to-size element is created;
g) cutting to size the flat combination of the wound dressing and cutting-to-size element produced in step f), wherein cutting to size is performed along the outline traced on the cutting-to-size element in step e);
h) positioning the cut-to-size liquid-permeable wound dressing on the wound;
i) covering the wound and the wound environment with the air-impermeable cover material of step a) so that the wound and the wound environment are closed in an air-tight manner;
j) connecting the element for functional connection of the wound space to a source of vacuum located outside the cover material; and
k) producing a vacuum of no more than 500 mm Hg in the wound space for at least 30 minutes.

15. The method for the vacuum therapy of wounds of claim 14, wherein, after the flat combination of the wound dressing and cutting-to-size element has been cut to size, the cutting-to-size element is removed from the cut-to-size liquid-permeable wound dressing before the cut-to-size wound dressing is placed on the wound.

16. A ready-to-use set for vacuum therapy of a wound, the set comprising:
an air-impermeable cover material for air-tight closure of the wound and of a wound environment;
an element for functional connection of a wound space to a source of vacuum located outside said cover material so that a vacuum can be established in the wound space and such that liquids can be suctioned out of the wound space;
a liquid-permeable wound dressing for inclusion in an interstice formed between a wound surface and said cover material;
a cutting-to-size element having a transparent film with a first and a second side, wherein said first side is structured to permit writing thereon, said second side having a self-adhesive coating; and
a package element within which said air-impermeable cover material, said element for functional connection of the wound space to a source of vacuum, said liquid-permeable wound dressing and said cutting-to-size element are accommodated in a sterilized condition thereof.
